(19) United States
(12) Patent Application Publication
(10) Pub. No.: US 2025/0312166 A1
(43) Pub. Date: Oct. 9, 2025

(54) WRIST IMPLANT GUIDE AND METHOD FOR ORIENTING A GUIDE WIRE FOR A WRIST JOINT REPLACEMENT

(71) Applicant: New York Society for the Relief of the Ruptured and Crippled, maintaining the Hospital for Special S, New York, NY (US)

(72) Inventors: Ryan Helbock, New York, NY (US); Scott Wolfe, New York, NY (US); Joseph Lipman, New York, NY (US)

(73) Assignee: New York Society for the Relief of the Ruptured and Crippled, maintaining the Hospital for Special S, New York, NY (US)

(21) Appl. No.: 19/170,744

(22) Filed: Apr. 4, 2025

Related U.S. Application Data
(60) Provisional application No. 63/575,387, filed on Apr. 5, 2024.
(60) Provisional application No. 63/631,746, filed on Apr. 9, 2024.

(51) Int. Cl. A61F 2/42; A61B 17/17; A61F 2/46

(52) U.S. Cl. CPC A61F 2/4261; A61B 17/1782; A61F 2/4606; A61F 2002/4677; A61F 2002/4687

(57) ABSTRACT

A device and method are described herein relating to a wrist implant guide assembly having a radial guide and a carpal guide. The radial guide may include a radial guide body having a patient-specific surface for matingly contacting a radial bone of a patient, and a radial guide wire aperture configured to direct a guide wire into a radial bone of the patient. The carpal guide may include a carpal guide body having a patient-specific surface for matingly contacting a capitate bone and a hamate bone of the patient, and a carpal guide wire aperture configured to direct a guide wire into a capitate bone of the patient.

### CROSS-REFERENCE TO RELATED APPLICATIONS

This patent application claims the benefit of U.S. Provisional Patent Application No. 63/575,387, entitled “PATIENT SPECIFIC GUIDES FOR WRIST ARTHROPLASTY,” filed Apr. 5, 2024, and U.S. Provisional Patent Application No. 63/631,746, entitled “PATIENT SPECIFIC GUIDES FOR WRIST ARTHROPLASTY,” filed Apr. 9, 2024, the contents each of which are incorporated by reference in their entireties as if fully set forth herein.

### FIELD OF THE INVENTION

The present invention relates to devices and methods useful for assisting in a wrist joint procedure, such as a wrist joint replacement procedure.

### BACKGROUND OF THE DISCLOSURE

A human body is made up of a variety of joints that interface with and join two or more bones. Such joints permit a variety of movements to occur at the joint between the two or more bones. A wrist joint, also referred to as the radiocarpal joint, include bones that communicate with one another to allow for a wide range of motions in the joint of the wrist. In particular, the wrist joint connects the forearm and the hand, and permits articulation between the radius and the carpal bones of the hand.

Different joint pathologies may occur in the bones or joints resulting from conditions such as osteoarthritis, or from traumas such as bone fractures, for example. A patient, or subject, with these joint pathologies may experience severe pain during movements of the joint resulting in severe disabilities due to limitations in joint movements. When severe joint pathologies occur, therapeutic methods such as the use of medications may not alleviate the pain and movement limitations in the joints of the patient. Partial or full joint replacement arthroplasty of the joint (e.g., wrist joint) may be the recommended course of treatment for the patient.

### SUMMARY OF THE DISCLOSURE

A wrist implant guide assembly is provided for assisting in wrist joint replacement procedures. The wrist implant guide assembly aids in orienting a guide wire during a surgical procedure involving implantation of a wrist joint. As described further herein, the wrist guide assembly may include a radial guide, a carpal guide, and/or a drill guide.

In accordance with an exemplary aspect of the subject disclosure, a wrist implant guide assembly may include a radial guide and a carpal guide. The radial guide may include a radial guide body having a patient-specific surface for matingly contacting a radial bone of a patient, and a radial guide wire aperture configured to direct a guide wire into a radial bone of the patient. The carpal guide may include a carpal guide body having a patient-specific surface for matingly contacting a capitate bone and a hamate bone of the patient, and a carpal guide wire aperture configured to direct a guide wire into a capitate bone of the patient. The patient-specific surface of the radial guide may matingly correspond to a distal portion of the radius bone of the patient.

In an aspect, the radial guide may include a dorsal flange extending from the radial guide body having a patient-specific surface for matingly contacting and covering a Lister's tubercle of the patient. The dorsal flange may include a plurality of stabilizer apertures. The plurality of stabilizer apertures may extend in non-parallel and non-perpendicular directions relative to each other.

In an aspect, the radial guide may include a guide wire housing extending from the radial guide body. The guide wire housing may include the radial guide wire aperture. The radial guide body may have a substantial planar distal surface from which a guide wire housing extends. The radial guide may include a plurality of peripheral radial guide wire apertures positioned peripherally about the radial guide wire aperture.

In an aspect, the carpal guide may include a carpal guide wire housing extending from the carpal guide body. The carpal guide wire housing may include the carpal guide wire aperture. The carpal guide may include a plurality of peripheral carpal guide wire apertures positioned peripherally about the carpal guide wire aperture.

In an aspect, the carpal guide may include an alignment arm for engaging a third metacarpal of the patient. The alignment arm may extend from the carpal guide body of the carpal guide.

In an aspect, the carpal guide body may include a stationary base that may be extendable from the carpal guide body. The stationary base may be engageable with a moveable arm of the alignment arm. The alignment arm may have a longitudinal length extending substantially parallel to an axis of the carpal guide wire aperture.

In an aspect, the wrist implant guide assembly may include a drill guide configured to engage the radial guide or the carpal guide. The drill guide may include a handle and a barrel extending therefrom. The barrel may include a plurality of guide wire apertures extending longitudinally therethrough. The barrel may have an overall longitudinal cross-sectional profile that is non-circular.

In an aspect, the radial guide may include a guide wire housing extending from the radial guide body and having a corresponding slot to receive the barrel of the drill guide therein, the barrel configured to engage the slot of the radial guide body. The carpal guide may include a guide wire housing extending from the carpal guide body and having a corresponding slot to receive the barrel of the drill guide therein, the barrel configured to engage the slot of the carpal guide body.

### BRIEF DESCRIPTION OF THE FIGURES

The foregoing summary, as well as the following detailed description of the exemplary embodiments of the subject disclosure, will be better understood when read in conjunction with the appended drawings. For the purpose of illustrating the present disclosure, exemplary embodiments are shown in the drawings. It should be understood, however, that the subject application is not limited to the precise arrangements and instrumentalities shown.

FIGS. 1A-D illustrate a radial guide in accordance with an exemplary embodiment of the subject disclosure;

FIGS. 1E-G illustrate the radial guide of FIG. 1A positioned upon an exemplary radius bone;

FIGS. 2A-C illustrate a radial guide in accordance with another embodiment of the subject disclosure positioned upon a radius bone;

FIGS. 3A-D illustrate a carpal guide in accordance with an exemplary embodiment of the subject disclosure;

FIGS. 3E-G illustrate the carpal guide of FIG. 3A positioned upon an exemplary capitate bone;

FIG. 4 illustrates a carpal guide in accordance with another embodiment of the subject disclosure positioned upon a capitate bone;

FIG. 5 illustrates a carpal guide in accordance with yet another exemplary embodiment of the subject disclosure positioned upon a capitate bone;

FIG. 6 illustrates a carpal guide in accordance with yet another exemplary embodiment of the subject disclosure positioned on a capitate bone;

FIGS. 7A-B illustrate a carpal guide in accordance with yet another exemplary embodiment of the subject disclosure;

FIGS. 8A-8C illustrate a radial guide positioned upon an exemplary radius bone in which the radial guide is configured to be used with a drill guide in accordance with another exemplary embodiment of the subject disclosure;

FIGS. 9A-9D illustrate a carpal guide positioned upon an exemplary radius bone in which the carpal guide is configured to be used with a drill guide in accordance with another exemplary embodiment of the subject disclosure; and

FIGS. 10A-10G illustrate a carpal guide in accordance with another embodiment of the subject disclosure.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one,” and “one or more,” as used in the specification, means at least one but may include any number from one to all.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

The human wrist is a complex joint that bridges the hand to the forearm. The wrist joint, also referred to as the radiocarpal joint, is a condyloid joint (e.g., ball and socket joint) of the distal upper limb that connects and serves as a transition point between the forearm and the hand. The wrist joint allows for flexion, extension, abduction, and adduction movements of the hand.

Patients may suffer from one or more ailments of the wrist (e.g., wrist joint). For example, patients may suffer from osteoarthritis of the wrist, or may have experienced traumatic injury or prior surgical complications with their wrist. Such patients may be candidates for arthroplasty of the wrist, such as total wrist arthroplasty (TWA). A surgical procedure may be performed that replaces the wrist joint with one or more implants. For example, a wrist procedure may be performed on a wrist by replacing the wrist joint with a radial implant and a carpal implant. The radial implant and the carpal implant may be configured to articulate with one another. Such wrist implant may result in the patient experiencing less pain in the wrist and a restored range of motion of the wrist.

In many cases wrist arthroplasty (e.g., total wrist arthroplasty) may have a high rate of complications, such as the implant loosening and the implant resulting in a limited range of motion of the wrist. To reduce the incidence of complications, and improve the success and durability of the device, an optimization in the radial and carpal implant orientation may be performed. Implant orientation may be dependent on the position of a guide wire that may be inserted into the radius and carpal bones. Despite the importance of implant orientation, current surgical techniques for inserting the guide wire may rely on generic guides, simple measurements that are difficult to perform on the patient, or the visual assessment of the surgeon performing the implant procedure. As a result, current surgical techniques may result in multiple attempts at guide wire placement being necessary, which may reduce operating time efficiency and may increase radiation exposure to the patient and medical staff due to the need for multiple fluoroscopy images. In additional, current surgical techniques may also result in a suboptimal orientation of the guide wire as well as the final implant.

The implant devices (e.g., guides) and methods described herein for using the implant guides may be used to achieve an optimal implant orientation and reduce operating room time. For example, one or more implant guides may be used to accurately and efficiently ensure that the guide wire is placed in a preoperatively planned position (e.g., an optimal position). The implant guides may be patient-specific and may articulate specifically with the patient's anatomy. The implant guides may include apertures for aligning the guide wire to the preoperative position(s).

As described herein, one or more patient-specific guides (e.g., radial guide and/or carpal guides) may be configured to orient implant components (e.g., a guide wire) during a total wrist arthroplasty. For example, the radial guide and/or the carpal guides described herein may provide an optimal orientation of the guide wire. The optimal placement of the guide wire may determine final implant orientation of wrist implant systems (e.g., total wrist implant systems). The radial and/or carpal guides may be manufactured (e.g., fabricated) to ensure that the guide wire and implant positions are aligned along one or more axes of the radius and carpal bones as part of surgical techniques and instrumentations.

One or more guides (e.g., radial guides) may be used for positioning a radial implant, one or more guides (e.g., carpal guides) may be used for positioning a carpal implant, and/or one or more drill guides may be used to assist one or both radial and carpal guides. In examples the guides may be 3D-printed. In some examples the guides may be formed of plastic material, although in other examples the guides may be formed of other materials, such as stainless steel and the like. The radial and carpal guides may have patient-specific features. The radial and carpal guides may include one or more patient-specific articular surfaces that may be placed in direct contact with the anatomy (e.g., exposed anatomy) of a patient's wrist during a TWA procedure.

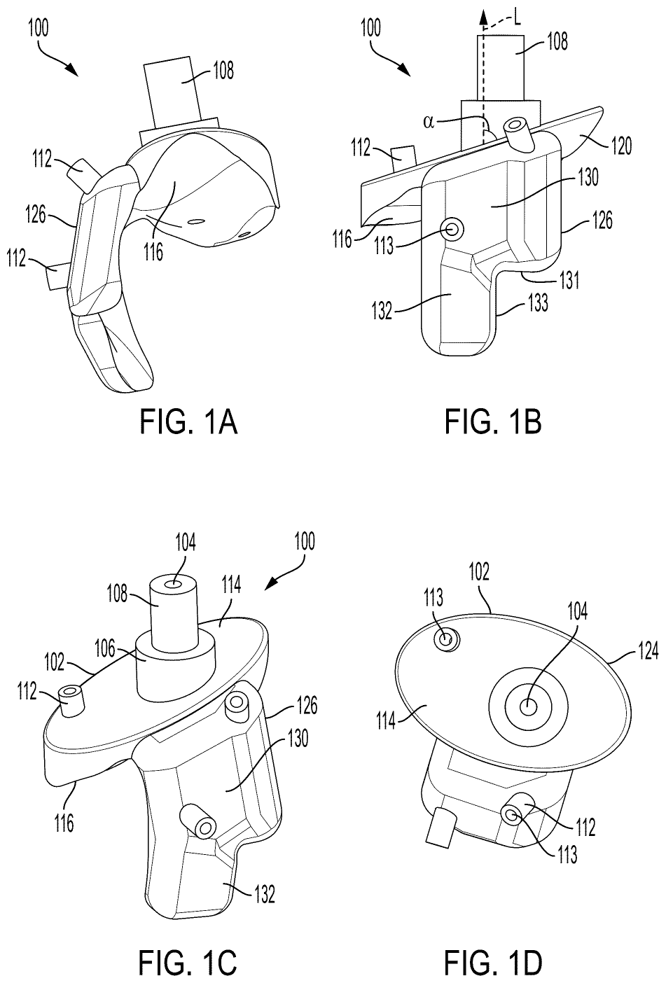

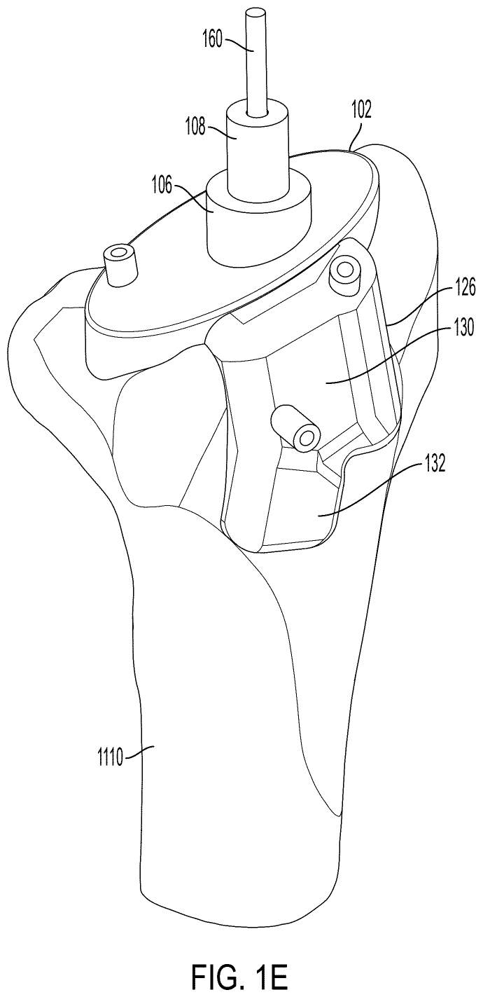

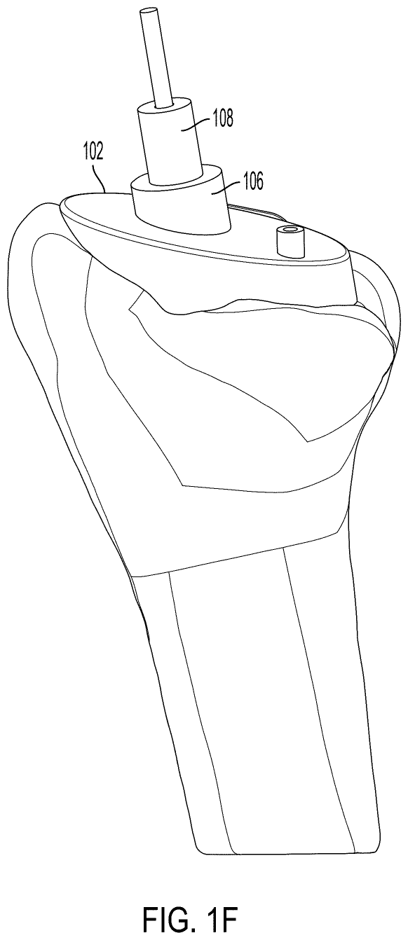

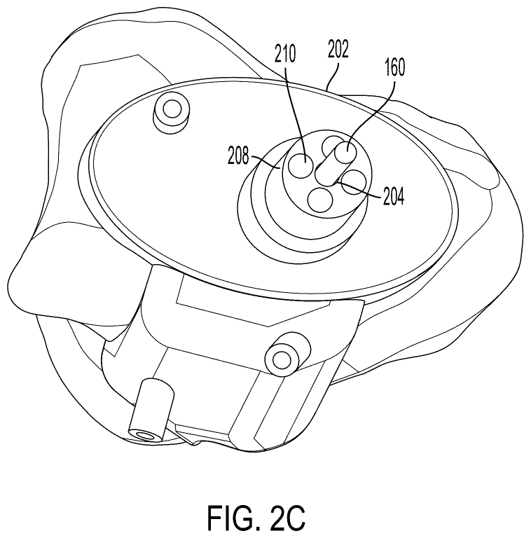

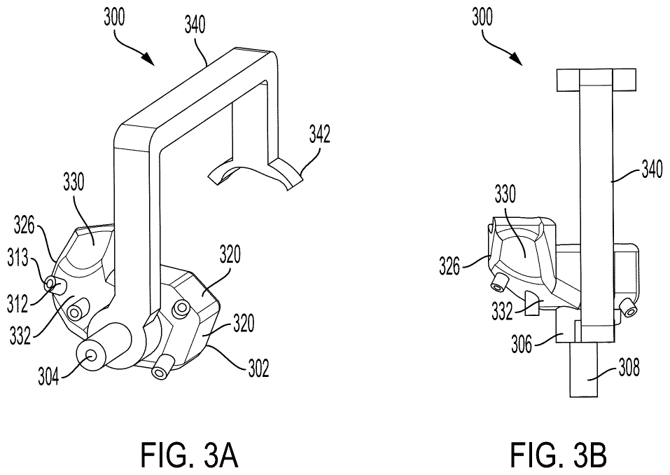

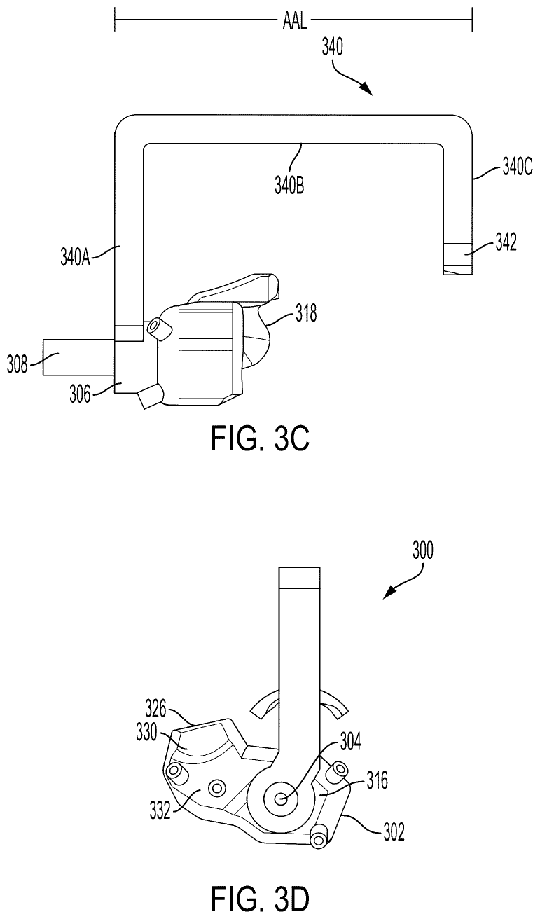

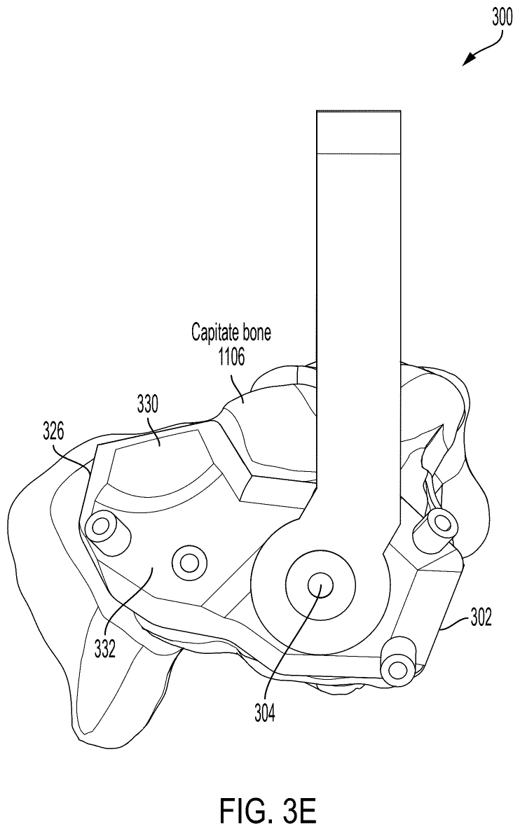

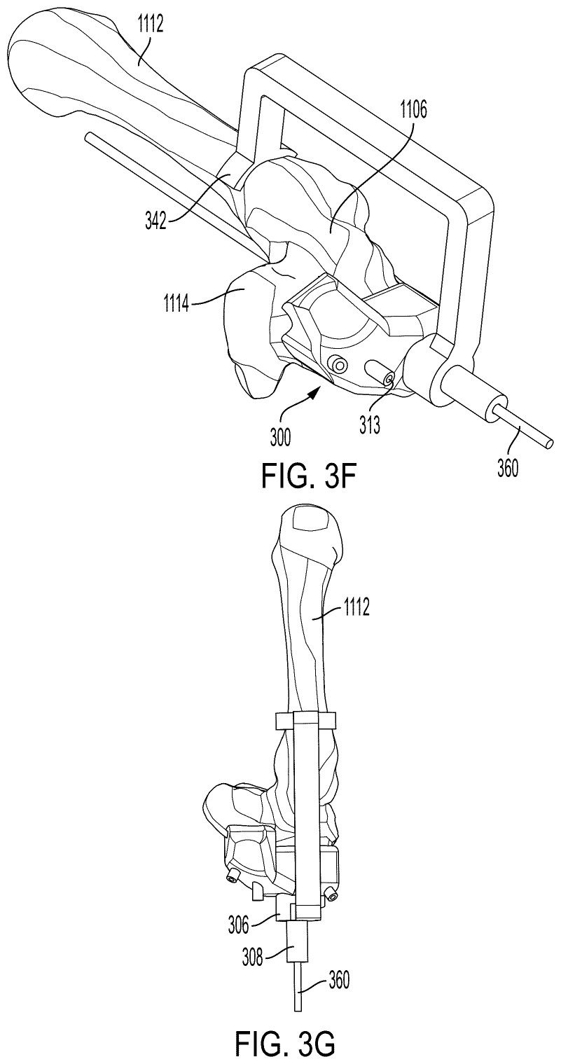

In accordance with an exemplary embodiment, the subject disclosure provides a wrist implant guide assembly comprising a radial guide 100 and a carpal guide 300, as best shown in FIGS. 1A-1G and 3A-3G, respectively.

Radial guide 100 is configured as shown in FIGS. 1A-1G. Radial guide 100 includes a radial guide body 102, a radial guide wire aperture 104, a dorsal flange 126, and a radial guide wire housing 108. Radial guide body 102 has a substantially oval shape and more particularly a substantially oval longitudinal cross-section at least along a portion of the radial guide body. The substantially overall shape of the radial guide body is an exemplary shape and other shapes may be applicable, such as circular, rectangular and the like. The radial guide body also includes a substantially planar distal end 114, and a proximal face 116 configured to matingly correspond to a patient's radial bone 1110.

As shown on FIGS. 1B and 1C, the radial guide body 102 may have one or more side walls or lateral walls 120 that extend from the substantially planar distal end 114. Lateral wall 120 may extend from the substantially planar distal end 114 in a curved or linear manner, although the configuration of the lateral wall may vary depending on the patient-specific bone anatomy to which the radial guide body is configured to matingly correspond to.

The substantially planar distal end 114 is configured as best shown in FIGS. 1C and 1D. While the distal end 114 is substantially planar, the distal end can alternatively be non-planar or of other configurations suitable for its intended purpose. The substantially planar distal end 114 is substantially planar throughout its entirety and defines a plane at an angle relative to a longitudinal axis of the radial guide 100. The angle may be based on the anatomy of the patient. In an embodiment, as best shown on FIG. 1B, the substantially planar distal end 114 may be at an angle α of about 40-80 degrees relative to the longitudinal axis L of the radial guide wire housing 108, including 40, 45, 50, 55, 60, 65, 70, 75 and 80 degrees.

As best shown on FIGS. 1A and 1C, the proximal face 116 has a surface configured to be a patient-specific surface. That is, the patient-specific surface of the proximal face 116 is configured to have a shape or contour that matingly corresponds to, contacts, and/or articulates with one or more bones of the patient, such as radial bone 1110 of the patient. The proximal face 116 may include grooves, slots, apertures, divots, protuberances, and the like so as to matingly correspond to and/or articulate with a radial bone 1110 of a specific patient.

The patient-specific surface of the proximal face 116 may be configured based on patient-specific images of the patient's wrist, such as pre-operative CT scans, radiographs, or other images of the patient's wrist. The pre-operative images are then utilized to generate three-dimensional models of the patient's bones and using CAD software and algorithms such as Boolean subtraction to model the patient-specific surface of the radial guide 100. FIGS. 1A-1C illustrate an exemplary patient-specific surface based on patient-specific images.

The dorsal flange 126 is configured as best shown in FIGS. 1A-1C and is located on or extends proximally from the radial guide body 102. Although FIGS. 1A-1C show a single dorsal flange 126, in alternative embodiments the radial guide can include a plurality of dorsal flanges that each extend from the radial guide body 102. Each of the dorsal flanges may include a plurality of stabilizer apertures 113.

Dorsal flange 126 may be shaped in a manner to matingly correspond to, contact, and/or articulate with a radial bone 1110 of the patient. As shown best on FIG. 1B, dorsal flange 126 is configured to cover or matingly engage the distal radial articular surface, including the radial styloid, the dorsal rim, and may partially or completely cover the Lister's tubercle. The dorsal flange may include a distal portion 130 and a proximal portion 132. The distal portion 130 may have a width that is greater than a width of proximal portion 132, although in examples the distal portion 130 and proximal portion 132 may have substantially similar widths or the distal portion 130 may have a width that is less than a width of the proximal portion 132. Referring to FIG. 1A, the distal portion 130 may extend proximally from the substantially planar distal end 114 at an angle based on the anatomy of the patient. For example, the distal portion 130 may extend proximally from the substantially planar distal end 114 at an angle of about 30-80 degrees relative to a longitudinal axis of the radial guide wire housing 108, including 30, 35, 40, 45, 50, 55, 60, 65, 70, and 75 degrees. The proximal portion 132 may extend from the distal portion at an angle based on the anatomy of the patient. For example, the proximal portion 132 may extend from the distal portion at an angle such as at about 10-60 degrees relative to a longitudinal axis of the distal portion 130, including 10, 15, 20, 25, 30, 35, 40, 45, 50, 55 and 60 degrees.

Dorsal flange 126 and radial guide body 102 may be formed as a unitary unit, although dorsal flange 126 may be configured as a separate part from radial guide body 102 and configured to couple (e.g., detachably couple) to radial guide body 102. Dorsal flange 126 may be comprised of a single piece or may include more than one piece. As shown in FIG. 1A, dorsal flange 126 is a concave curved flange that extends proximally from the substantially planar distal end 114. In exemplary embodiments, the dorsal flange may be linearly shaped or may take any other shape or form that allows the dorsal flange to matingly correspond to, contact, and/or articulate with a radial bone 1110 of the patient.

The radial guide wire housing 108 is configured as best shown in FIGS. 1A-1C and extends from the substantially planar distal end 114. Radial guide wire housing 108 may be annularly shaped, although in alternative examples the radial guide wire housing may be alternatively shaped, such as a square, rectangle, oval, or any other suitable shape for its intended purpose. The radial guide body 102 may have a substantial planar distal surface from which the guide wire housing 108 extends. In other words, radial guide wire housing 108 may include or extend from one or more housing steps, such as radial guide wire housing step 106. Radial guide wire housing step 106 is shown on FIGS. 1A-1C. Radial guide wire housing step 106 may be annularly shaped, although in examples the radial guide wire housing step may be alternatively shaped, such as a square, rectangle, oval, or any other suitable shape for its intended purpose. Radial guide wire housing 108 may include one or more radial guide wire apertures 104. Radial guide wire aperture 104 is configured to accept one or more guide wires, such as guide wire 160 (FIGS. 1E, 1F). In other words, the radial guide 100 may include a guide wire housing 108 extending from the radial guide body 102. The guide wire housing 108 may include the radial guide wire aperture 104.

Radial guide may have one or more stabilizer apertures or pin holes 113. As shown on FIGS. 1A-1G, stabilizer apertures 113 may be formed from annular channels 112, although in examples stabilizer apertures 113 may not be formed from annular channels 112. For example, stabilizer apertures 113 may not extend from a surface and instead may be flat to the surface in which stabilizer aperture 113 is located. One or more stabilizer apertures 113 and respective annular channels 112 may be positioned on one or more of substantially planar distal end 114, dorsal flange 126, and side wall 120. Stabilizer aperture 113 may be configured to receive a stabilizing device, such as a k-wire pin or other device for stabilizing radial guide 100 upon a patient. The one or more stabilizer apertures 113 and respective annular channels 112 may be located in one or more positions about a radial guide body 102 so as to best stabilize radial guide 100 to the patient. The stabilizer apertures 113 being extruded may provide a more controlled trajectory of the stabilizer apertures 113 and/or may minimize or prevent the stabilizer wires from interfering with each other inside the bone of the patient.

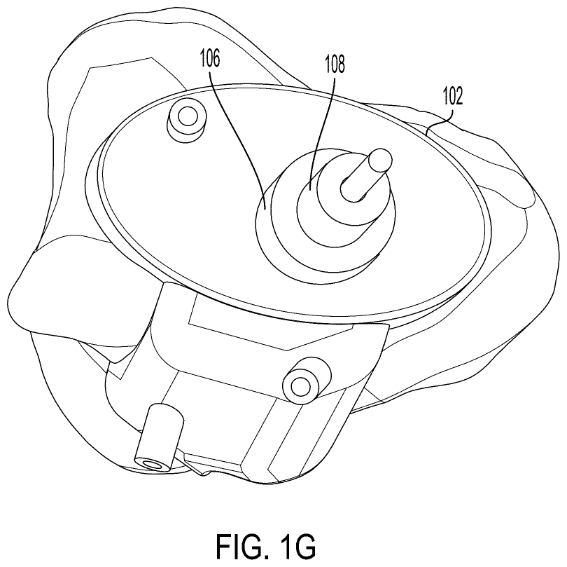

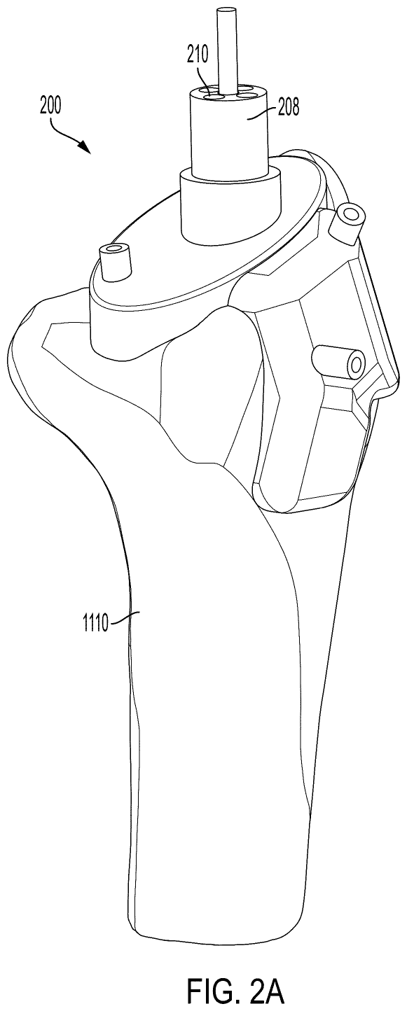

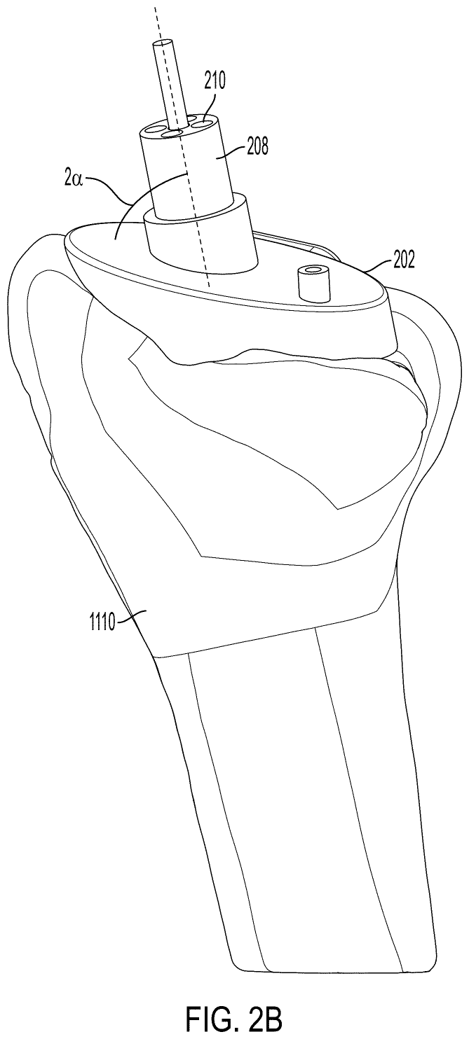

FIGS. 2A-2C illustrate another exemplary embodiment of a radial guide, such as radial guide 200. Radial guide 200 is substantially the same as radial guide 100, except as further discussed and described below. In this embodiment, a radial guide wire housing 208 includes a plurality of peripheral radial guide wire apertures 210 for receiving one or more guide wires. The radial guide 200 may include a plurality of peripheral radial guide wire apertures 210 positioned peripherally about the radial guide wire aperture 204. The plurality of peripheral apertures 210 may extend in a parallel direction relative to radial guide wire aperture 204. The radial guide wire housing 208 may include e.g., four apertures circumferentially spaced about a radial guide wire aperture 204. Alternatively, the radial guide wire housing 208 may include 1, 2, 3, 5, or 6 apertures circumferentially spaced about the radial guide wire aperture 204. Each of the plurality of peripheral radial guide wire apertures 210 are arranged to have a longitudinal axis substantially parallel with a longitudinal axis of the radial guide wire aperture 204 and at about an angle 2Ε based on the anatomy of the patient. For example, angle 2α may be about 30-60 degrees relative to a planar distal face of the radial guide body 202, including 35, 40, 45, 50, and 55 degrees.

FIGS. 2A-2C show peripheral radial guide wire apertures 210 being located on radial guide wire housing 208, although such location is for illustration purposes and the location of peripheral apertures 210 should not be so limiting. For example, radial guide 200 may alternatively include one or more peripheral radial guide wire apertures that may be located on one or more locations about the radial guide.

Carpal guide 300 is configured as shown in FIGS. 3A-3G. Carpal guide 300 includes a carpal guide body 302 having a patient-specific surface for matingly contacting a capitate bone 1106 and/or a hamate bone 1114 of the patient, a carpal guide wire aperture 304, and a lateral flange 326. The carpal guide wire aperture 304 is configured to direct a guide wire into a capitate bone 1106 of the patient. As best shown in FIG. 3D, carpal guide body 302 includes a substantially planar proximal end 316, and FIG. 3C shows carpal guide body 302 including a distal face 318 configured to matingly correspond to a patient's capitate bone 1106.

The substantially planar proximal end 316 is configured as best shown in FIGS. 3A-3F. While the proximal end 316 is substantially planar, the proximal end can alternatively be non-planar or of other configurations suitable for its intended purpose.

As best shown on FIGS. 3C and 3E-3G, the carpal guide body 302 has a distal face 318 including a surface configured to be a patient-specific surface. That is, the patient-specific surface of the distal face 318 is configured to have a shape or contour that matingly corresponds to, contacts, and/or articulates with one or more bones of the patient, such as capitate bone 1106 or hamate bone 1114 of the patient. The patient-specific surface may be configured based on patient-specific images of the patient's wrist, such a pre-operative CT scans, radiographs, or other images of the patient's wrist. The pre-operative images may be utilized to generate three-dimensional models of the patient's bones using CAD software and algorithms such as Boolean subtraction to model the carpal guide body 302 (e.g., patient-specific surface of the distal face 318) of the capitate guide 300.

As shown on FIG. 3A, the carpal guide body 302 may have one or more lateral walls 320 that extend distally from the substantially planar proximal end 316. Lateral walls 320 may extend from the substantially planar proximal end 316 in a straight or linear manner, while in examples lateral walls may extend from the substantially planar proximal end 316 in a curved or non-linear manner. The configuration of the lateral walls may vary depending on the patient-specific bone anatomy to which the carpal guide body is configured to matingly correspond to. The lateral walls 320 may also have an interior wall or bone engaging surface that is a patient-specific bone engaging surface. The lateral walls 320 may also be configured to circumscribe bone or be segmented such that each segment of the lateral walls are at an angle relative to each other.

The lateral flange 326 is configured as best shown in FIGS. 3B-3E and is located on or extends distally from the carpal guide body 302. Although FIGS. 3B-3E show a single lateral flange 326, in alternative embodiments the carpal guide can include a plurality of lateral flanges that each extend from the carpal guide body 302. Lateral flange 326 may be shaped in a manner to matingly correspond to, contact, and/or articulate with a capitate bone 1106 and/or hamate bone 1114 of the patient. As shown best on FIGS. 3C and 3E, lateral flange 326 is configured to cover or matingly engage the proximal end of the capitate bone 1106 and/or hamate bone 1114, as well as part of the dorsal surfaces of those bones. The lateral flange includes a lateral flange distal portion 330 and a lateral flange proximal portion 332.

The lateral flange proximal portion 332 may have a width that is greater than a width of the lateral flange distal portion 330, although in examples the lateral flange proximal portion 332 and the lateral flange distal portion 330 may have substantially similar widths or the lateral flange proximal portion 332 may have a width that is less than a width of the lateral flange distal portion 330.

Referring to FIG. 3B, the lateral flange proximal portion 332 may extend distally from the substantially planar proximal end 316 at an angle based on the anatomy of the patient, such as at angle of about 30-80 degrees relative to a longitudinal axis of the carpal guide body 302, including 30, 35, 40, 45, 50, 55, 65, 70, and 75 degrees. For example, the outer surface of the lateral flange proximal portion may extend distally from the substantially planar proximal end at an angle of about 30-80 degrees relative to a longitudinal axis of the carpal guide body 302, including 30, 35, 40, 45, 50, 55, 65, 70, and 75 degrees. The lateral flange distal portion 330 may extend from the lateral flange proximal portion 332 at an angle based on the anatomy of the patient, such as at an angle of about 10-60 degrees relative to a longitudinal axis of the lateral flange proximal portion 330, including 10, 15, 20, 25, 30, 35, 40, 45, 50, 55 and 60 degrees. For example, the outer surface of the lateral flange distal portion 330 may extend from the outer surface of the lateral flange proximal portion 332 at an angle of about 10-60 degrees relative to a longitudinal axis of the outer surface of the lateral flange proximal portion 330, including 10, 15, 20, 25, 30, 35, 40, 45, 50, 55 and 60 degrees.

Lateral flange 326 and carpal guide body 302 may be formed as a unitary unit, although lateral flange 326 may be configured as a separate part from carpal guide body 302 and configured to couple (e.g., detachably couple) to carpal guide body 302. Lateral flange 326 may be comprised of a single piece or may be comprised of a plurality of pieces connectable together.

The carpal guide wire housing 308 is configured as best shown in FIGS. 3A and 3B and extends from the proximal end 316. Carpal guide wire housing 308 may be annularly shaped, although in alternative examples the carpal guide wire housing may be alternatively shaped, such as a square, rectangle, oval, or any other suitable shape for its intended purpose. The carpal guide 300 may include a carpal guide wire housing 308 extending from the carpal guide body 302. The carpal guide body 302 may include the carpal guide wire aperture 304. Carpal guide wire housing 308 may include or extend from one or more housing steps, such as carpal guide wire housing step 306. Carpal guide wire housing step 306 is shown best on FIGS. 3A-3C. Carpal guide wire housing step 306 may be annularly shaped. Carpal guide wire housing 308 may include one or more carpal guide wire apertures 304. Carpal guide wire aperture 304 is configured to accept a guide wire, such as guide wire 360 (FIGS. 3F, 3G).

Carpal guide body 302 may have one or more stabilizer apertures or pin holes 313. As shown on FIGS. 3A-3G, stabilizer apertures 313 may be formed as an annular member 312. Alternatively, instead of annular members, the stabilizer apertures may be formed as through holes on the carpal guide. One or more stabilizer apertures may be positioned on one or more of the proximal end, the lateral flange, and the lateral walls. Stabilizer aperture 313 may be configured to receive a stabilizing device, such as a k-wire pin or other device for stabilizing carpal guide 300 upon a patient. The one or more stabilizer apertures 313 may be located in one or more positions about a carpal guide body 302 and have central longitudinal angles at various angles relative to each other so as to best stabilize carpal guide 300 to the patient.

As shown on FIGS. 3A-3G, carpal guide 300 may include an alignment arm 340. Alignment arm 340 is configured to engage with a third metacarpal of a patient. Alignment arm 340 extends from the carpal guide body 302 of carpal guide 300 or is attachable to the carpal guide body 302 of carpal guide 300. The alignment arm 340 is a substantially C-shaped arm that extends distally from the carpal guide. The alignment arm includes a first segment 340A that is connected to or attachable to the carpal guide body 302, a second segment 340B that extends distally from the first segment, and a third segment 340C that extends in a direction transverse to a direction the second segment extends. The third segment 340C is configured to extend towards and engage a bone of the patient, such as the third metacarpal 1112 (FIG. 3F) of the patient. The third segment 340C also includes an alignment hand 342 configured to engage a bone of the patient, such as a 3^{rd} metacarpal 1112 of the patient. As shown on FIG. 3F, the alignment hand 342 is configured to include a curved or recessed paw to fixedly or contactingly engage with a bone of the patient, although alignment hand may be shaped in different forms (e.g., jagged, linear, including protuberances, including apertures, etc.) as needed to engage with a bone of the patient.

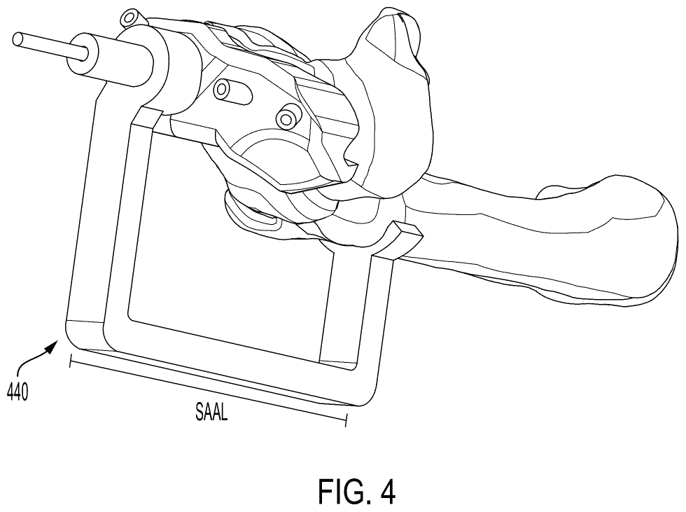

In an exemplary embodiment, the alignment hand 342 may include a bone-mating surface that is patient-specific. The bone-mating surface may be configured so the surgeon can skeletonize the base of the 3rd metacarpal 1112 of the patient and seat the bone-mating surface of the alignment hand 342 directly onto the 3rd metacarpal 1112 of the patient. As a result, the bone-mating surface of the alignment hand 342 may rest (e.g., directly rest) on the 3rd metacarpal 1112 of the patient, as shown in FIG. 3F and FIG. 4. In other embodiments, the alignment hand 342 may not have a bone-mating surface. In such embodiments the surgeon would not skeletonize the 3rd metacarpal 1112 of the patient and the alignment hand 342 would rest over the skin of the patient. For example, FIG. 5 shows alignment hand 342 does not have the bone-mating surface and does not contact (e.g., directly contact) the bone of the patient.

The alignment arm 340 may have a longitudinal length AAL (FIG. 3C) with the second segment extending substantially parallel to an axis of the guide wire aperture 308. The second segment is also configured to run substantially parallel to a longitudinal axis of the 3rd metacarpal 1112 of the patient. That is, the guide wire aperture 308 is configured to substantially align with a longitudinal axis of the 3^{rd} metacarpal of a patient's bone. For example, in an embodiment the second segment may be angled 5-10 degrees with respect to the 3^{rd} metacarpal of the patient's bone.

In alternative aspects, the alignment arm may be configured to have differing lengths. For example, FIG. 4 shows an alternative embodiment where the alignment arm 440 has a length SAAL, which is shorter than an overall length of the alignment arm 340. FIG. 5 shows an alternative embodiment where the alignment arm 540 has a length LAAL, which is greater than an overall length of the alignment arm 340. The alignment arm can be configured to have an overall length that is sufficient for the alignment hand to engage a proximal end of a patient's metacarpal, a midportion of the patient's metacarpal, or a distal end of the patient's metacarpal.

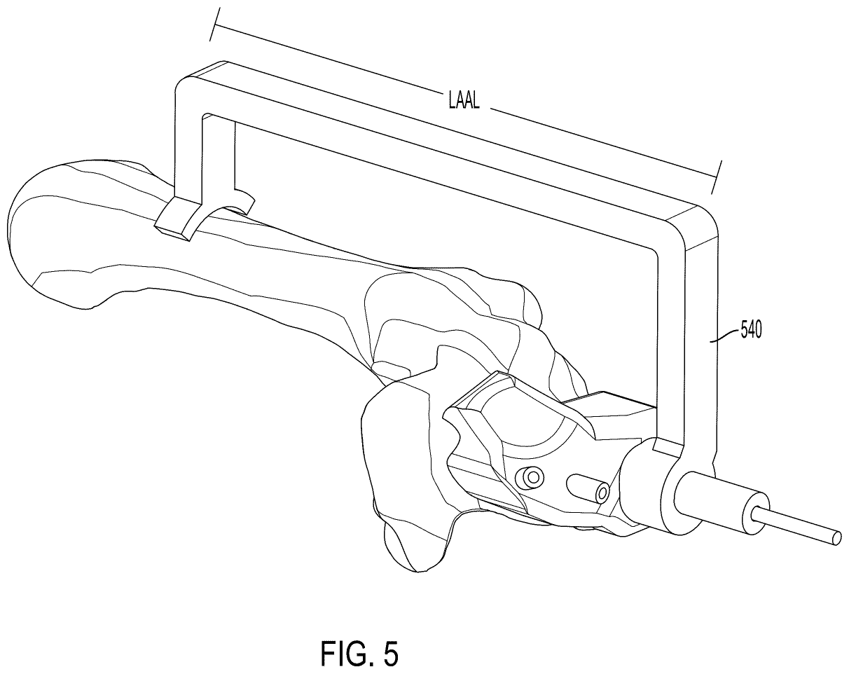

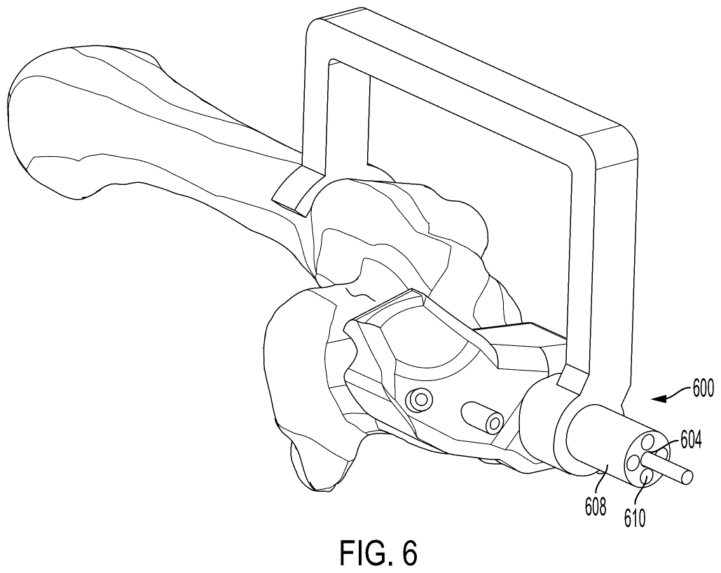

FIG. 6 shows an exemplary embodiment of carpal guide 600 that includes a plurality of guide wire apertures, such as peripheral carpal guide wire apertures 610, for receiving a guide wire. Carpal guide 600 is substantially the same as carpal guide 300 discussed above except as expressly described herein. Peripheral carpal guide wire apertures 610 may be positioned about the carpal guide wire aperture 604. In other words, the plurality of peripheral carpal guide wire apertures 610 may be positioned peripherally about the carpal guide wire aperture 604. Peripheral carpal guide wire apertures 610 are located on carpal guide wire housing 608. In other alternative embodiments a carpal guide may include one or more peripheral radial guide wire apertures that may be located on one or more locations about a carpal guide. The plurality of guide wires apertures are configured to each have central longitudinal axis that is substantially parallel to the carpal guide wire aperture 604.

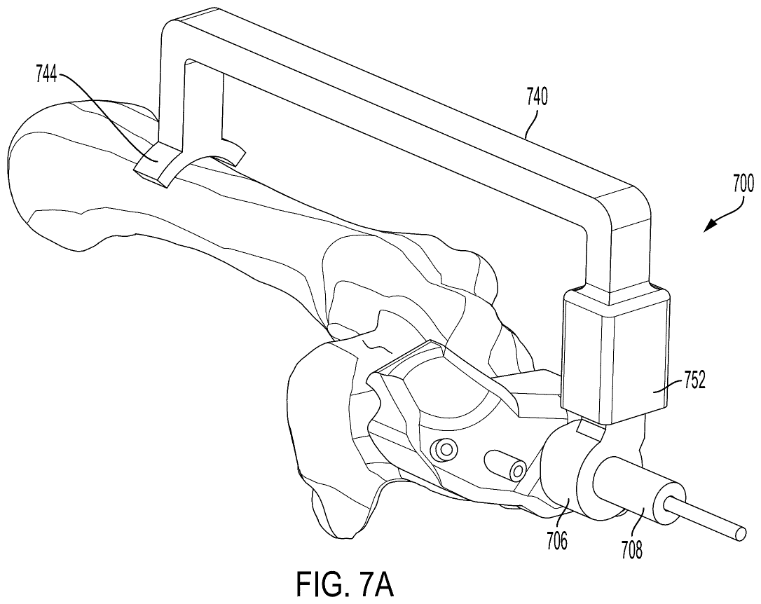

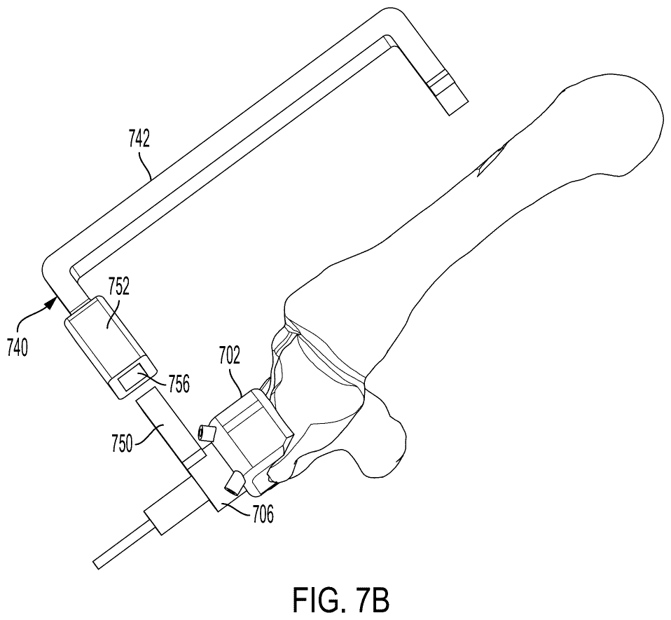

FIGS. 7A-7B illustrate another exemplary embodiment of carpal guide 700 in which alignment arm 740 is movable and/or detachable. Alignment arm 740 is configured to engage with a third metacarpal of a patient. The carpal guide body 702 includes a stationary base 750. Stationary base 750 may be engageable with or attached to the alignment arm 740. In particular, stationary base 750 may extend from the carpal guide body 702 and engage with or attach to the alignment arm 740.

Moveable arm 742 of the alignment arm may include a base receiving portion 752 for receiving stationary base 750. Moveable arm 742 may extend from and be movable relative to the stationary base 750. Base receiving portion 752 may include one or more base apertures 756 (FIG. 7B). As shown on FIG. 7B, stationary base 750 may be formed of flat sides (e.g., four flat sides) and base apertures 756 of base receiving portion 752 may be sized and shaped to receive and engage the particular size and shape of stationary base 750. Although FIG. 7C shows a stationary base 750 with four flat sides, such embodiment is for illustration purposes. Other embodiments may include stationary base 750 having two or more sides that may be flat, curved, round, and the like. The attachable moveable arm 742 advantageously allows for different sized arms to be attached to the carpal guide.

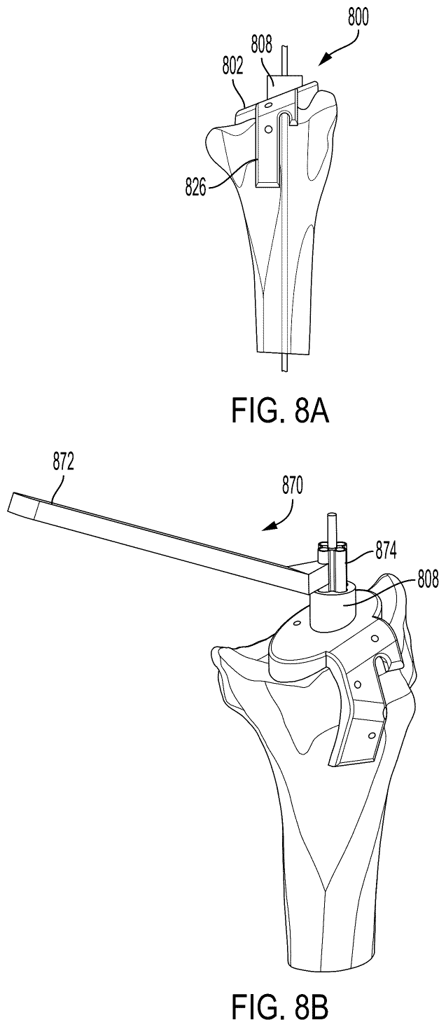

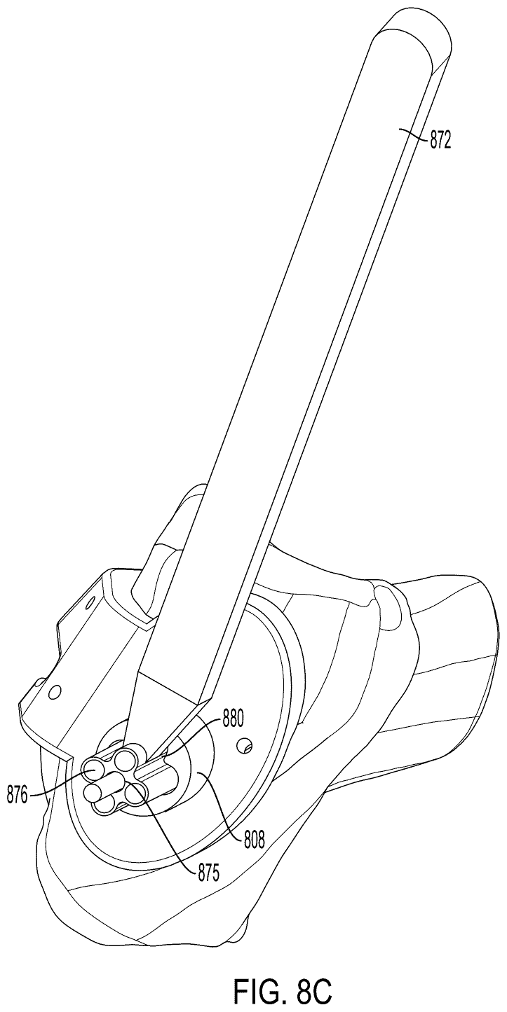

FIGS. 8A-8C illustrate another exemplary embodiment of a radial guide 800 in accordance with the subject disclosure. In this embodiment a wrist implant guide assembly may include a radial guide using a drill guide and/or a carpal guide using drill guide. In other words, a drill guide may be configured to engage the radial guide or the carpal guide. The drill guide for the radial guide and the carpal guide may be the same drill guide, although in other embodiments the drill guide for the radial guide and the carpal guide may be drill guides of differing configurations.

The drill guide 870 is configured to engage the radial guide 800. The drill guide 870 includes a handle 872 and a barrel 874 extending from the handle 872. In an exemplary embodiment, the barrel 874 may be configured to engage the radial guide 800. The barrel 874 may have one or more guide wire apertures 875, 876. In an exemplary embodiment, the drill guide 870 may have a plurality of guide wire apertures 875, 876 extending longitudinally therethrough. As shown on FIG. 8B the barrel 874 of drill guide 870 may include a guide wire aperture 875 that is centered and one or more guide wire apertures 876 that are peripheral to the guide wire aperture 875 that is centered. The guide wire apertures 875, 876 may extend longitudinally through the barrel 874.

The radial guide 800 includes a guide wire housing 808 that extends from the radial guide body 802. The radial guide wire housing 808 includes a slot 880. Slot 880 is configured to receive the drill guide 870 (e.g., barrel 874 of drill guide 870). In particular, the barrel 874 is configured to engage the slot 880 of the radial guide body 802 (e.g., guide wire housing 808 of radial guide body 802). The barrel 874 may have an overall longitudinal cross-sectional profile that is non-circular prevents the drill guide 870 (e.g., the barrel 874 of the drill guide 870) from rotating when the barrel 874 of the drill guide 870 is engaged with the slot 880 of the radial guide 800. In other words, the radial guide 800 may include a guide wire housing 808 extending from the radial guide body 802 and having a corresponding slot 880 to receive the barrel 874 therein. The barrel 874 may be configured to engage the slot 880 of the radial guide body 802.

The radial guide body 802 is configured substantially the same as radial guide body 102 except that the dorsal flange 826 is configured as best shown in FIG. 8A to include spaced flange segments. The articular surface of the radial guide body is also configured to contact the scaphoid/lunate fossa surfaces of the distal radius, wrap over the dorsal rim of the radius, and contact the dorsal surface of the radius, specifically straddling Lister's tubercle, or any other prominent feature found on the dorsal surface.

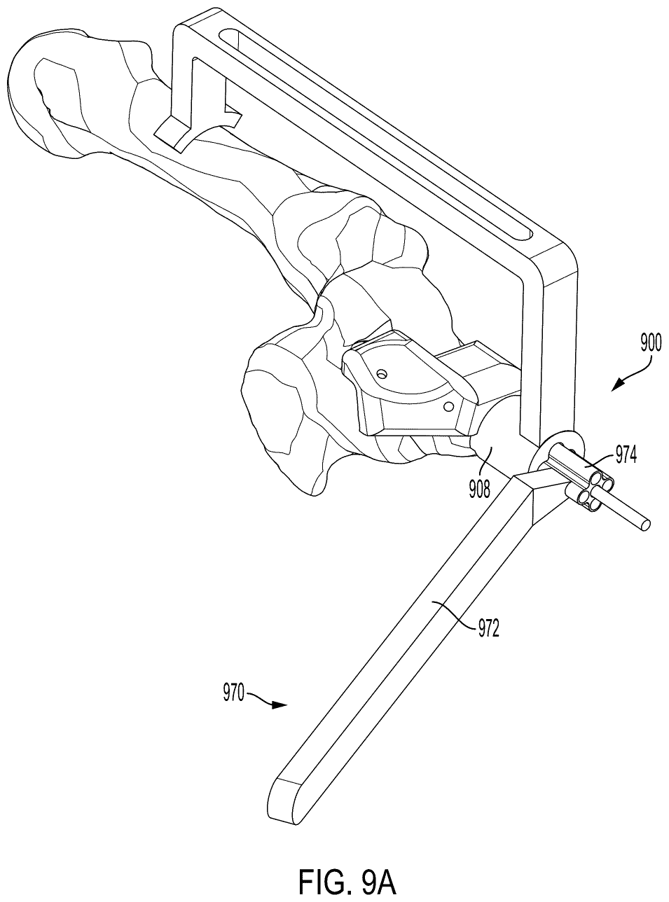

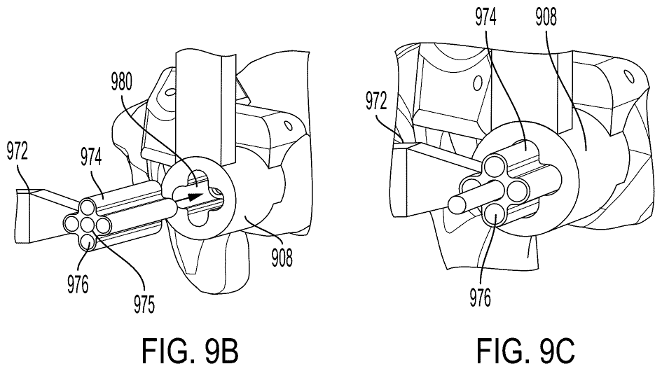

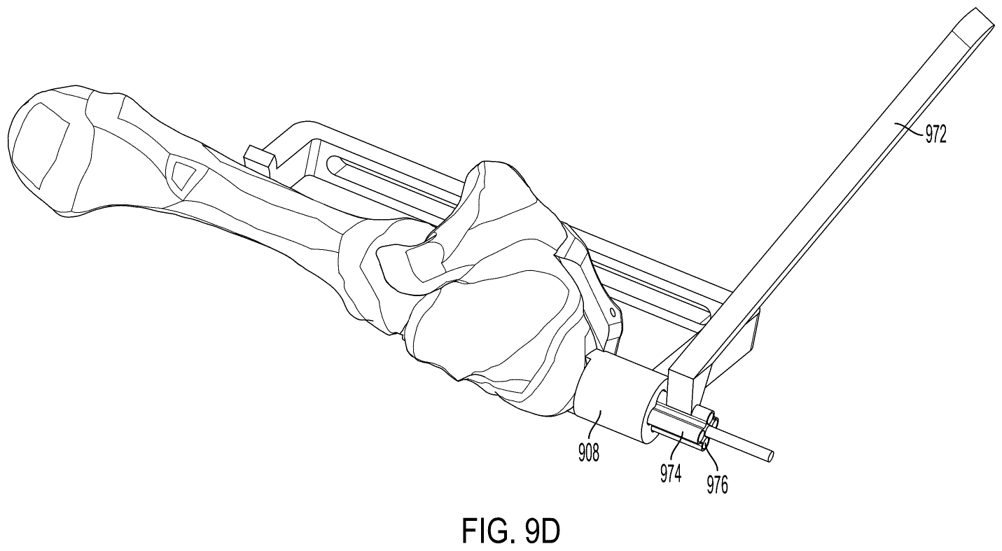

FIGS. 9A-9D illustrate another exemplary embodiment of a drill guide 970 configured to engage a carpal guide 900. The drill guide 970 includes a handle 972 and a barrel 974 extending from the handle 972. The barrel 974 may include one or more guide wire apertures 975, 976. In an exemplary embodiment, the barrel 974 of the drill guide 970 may include a guide wire aperture 975 that is centered upon one or more guide wire apertures 976 that are peripheral to the guide wire apertures 975 that is centered. The guide wire apertures 975, 976 may extend longitudinally through the barrel 974. The barrel 974 has an overall longitudinal cross-sectional profile that is non-circular.

The carpal guide 900 includes a guide wire housing 908 that extends from the carpal guide body 902. The carpal guide wire housing 908 includes a slot 980. Slot 980 is configured to receive the drill guide 970 (e.g., barrel 974 of drill guide 970). In particular, the barrel 974 is configured to engage the slot 980 of the carpal guide body 902 (e.g., guide wire housing 908 of carpal guide body 902). The barrel 974 having an overall longitudinal cross-sectional profile that is non-circular prevents the drill guide 970 (e.g., the barrel 974 of the drill guide 970) from rotating when the barrel 974 of the drill guide 970 is engaged with the slot 980 of the carpal guide 900. In other words, the carpal guide 900 may include a guide wire housing 908 extending from the carpal guide body 902 and having a corresponding slot 980 to receive the barrel 974 therein. The barrel 974 may be configured to engage the slot 980 of the carpal guide body 902.

Examples of the radial guide and carpal guide may be used with a drill guide or without a drill guide. FIGS. 8A-8C show a radial guide 800 using a drill guide 870. FIGS. 9A-9D show a carpal guide 900 using a drill guide 970. FIGS. 1A-1G and FIGS. 2A-2C show respective radial guides 100, 200 without the use of a drill guide. FIGS. 3A-3G, FIG. 4A, FIG. 5A, FIG. 6A, and FIGS. 7A-7B show carpal guides 300, 400, 500, 600, and 700 without the use of a drill guide.

The patient specific surfaces (e.g., articular surfaces) of the guides may be created with the use of CAD software. That is, the patient-specific bone models may be obtained by 3D reconstruction of a patient's preoperative CT imaging scans using algorithms such as Boolean subtraction to model the patient specific surfaces (e.g., articular surfaces) of the guides.

One or more of the implant guides (e.g., radial guide, carpal guides) may include one or more apertures configured to direct a guide wire into the bone of patient in a preferred orientation (e.g., an orientation determined during preoperative planning). The implant guides (e.g., radial guide, carpal guide) may include one or more other apertures to stabilize the guides upon the wrist. Such stabilizing apertures may include apertures for receiving k-wires or other surgical devices which may allow the guides to be affixed and/or pinned to the bone of the patient for improved stabilization.

As described herein, the surfaces (e.g., articular surfaces) of the implant guides (e.g., radial guide, carpal guide) may be patient-specific and thus may differ from patient to patient. The area that the articular surface (e.g., articular surface of the implant guides) will cover may differ between designs based on the features on the patient's anatomy that provides adequate fixation for the guide. For example, with respect to the radial guide, the articular surface may cover a larger or smaller area of the scaphoid/lunate fossa surface of the distal radius of the patient, which may extend toward the radial styloid. The surface of the implant guides may cover a larger portion of the dorsal rim and dorsal surface of the patient. The flanges on the dorsal surface may be longer/shorter or wider/thinner in examples and may rotate about the axis of the radius of the patient depending on where the Lister's tubercle or other prominent features of the patient are located. For example, depending on the amount of tissue release required for the patient, the surgeon may completely skeletonize the Lister's tubercle. In examples the surgeon may perform a partial tissue release by only skeletonizing the distal half of Lister's tubercle.

In examples in which the surgeon completely skeletonizes the Lister's tubercle, the radial guide shown on FIGS. 1A-1G may be used. In examples in which the surgeon performs a partial tissue release by only skeletonizing the distal half of Lister's tubercle, the radial guide may require a modification to the dorsal flange such that it would only cover the distal half of Lister's tubercle. The surface (e.g., articular surface) of the carpal guide may be modified on a patient-specific basis. For example, the surface of the carpal guide may be modified on a patient-specific basis to cover more or less of the capitate and hamate of the patient by extending further/shorter along the dorsal, volar, medial, and/or lateral surfaces of the patient.

The drill guide used with the radial guide and the drill guide used with the carpal guide may be the same and may be used (e.g., may be used interchangeably) with both the radial guide and the carpal guide, although in examples the drill guides used for the radial guide and carpal guide may be different. The drill guide may be separate from the radial guide and/or the carpal guide, although in examples the drill guide may be formed from radial guide and/or carpal guide. The drill guide may be designed to insert into one or more portions of the radial and/or carpal guides. The portion of radial guide and/or carpal guide in which drill guide engages, such as slot, may take many different and various forms. For example, the portion of radial guide and/or carpal guide in which drill guide engages may be circular or non-circular.

The corresponding barrel of drill guide that engages with the radial guide and/or carpal guide may be of a similar shape as the portion of radial guide and/or carpal guide in which drill guide engages. For example, the portion of radial guide and/or carpal guide in which drill guide engages, and the corresponding barrel of drill guide that engages with the radial guide and/or carpal guide, may be circular or non-circular. In examples in which the carpal guide and corresponding barrel are non-circular, such configuration may eliminate rotational freedom of the drill guide when drill guide is engaged with the carpal guide or radial guide.

Drill guide may be configured to be of size and/or length to properly direct the guide wire in the desired location (e.g., the preoperatively determined orientation). For example, the barrel of drill guide may be used to direct guide wire in the desired location of the patient. In examples, the drill guide may alleviate obstructions in radial guide and/or carpal guide for placement of the guide given the small operating space within a wrist of a patient. In examples in which drill guide is used, radial guide and/or carpal guide may first be placed on the anatomy of the patient and drill guide may be inserted into the corresponding slot of carpal guide and/or the slot of the radial guide.

The drill guide may include one or more guide wire apertures, which may include a centered guide wire aperture as well as peripheral guide wire apertures. Peripheral guide wire apertures on the drill guide may be located proximate to the centered guide wire aperture. Peripheral guide wire apertures may be located anywhere on drill guide, such as positioned radially around the centered guide wire aperture. Peripheral guide wire apertures may provide additional options for orienting guide wire, which may provide flexibility within the operating room. For example, peripheral guide wire apertures may provide additional options for orienting the guide wire if a surgeon encounters a situation where the position of the guide wire is required to be shifted. In examples in which drill guide is separate from the radial guide and carpal guide, the drill guide may include patient-agnostic features and the drill guide may be manufactured separately from the radial and/or carpal guide. In such examples, drill guide may be manufactured from different material (e.g., metal) than the radial and/or carpal guide, which may provide additional strength to the drill guide.

As described herein, the radial and carpal guides may provide desired positioning and/or orientation of a guide wire. Surfaces (e.g., patient-specific articular surfaces) of the radial and/or carpal guides may allow the guides to be positioned directly on the anatomy of each patient and provide accurate and efficient guide wire placement within each patient. Because the radial and carpal guides may be created and positioned according to each patient, the guides may be designed to provide any orientation deemed appropriate for the patient during preoperative planning. In other words, the guides of the subject disclosure provide accurate and efficient placement of the guide wire that will determine the orientation of the final implants of a total wrist arthroplasty. Existing technology is generic, based on externally-visible anatomic landmarks, and inefficient leading to suboptimal positioning of the TWA implants, extended operating time, and increased radiation exposure.

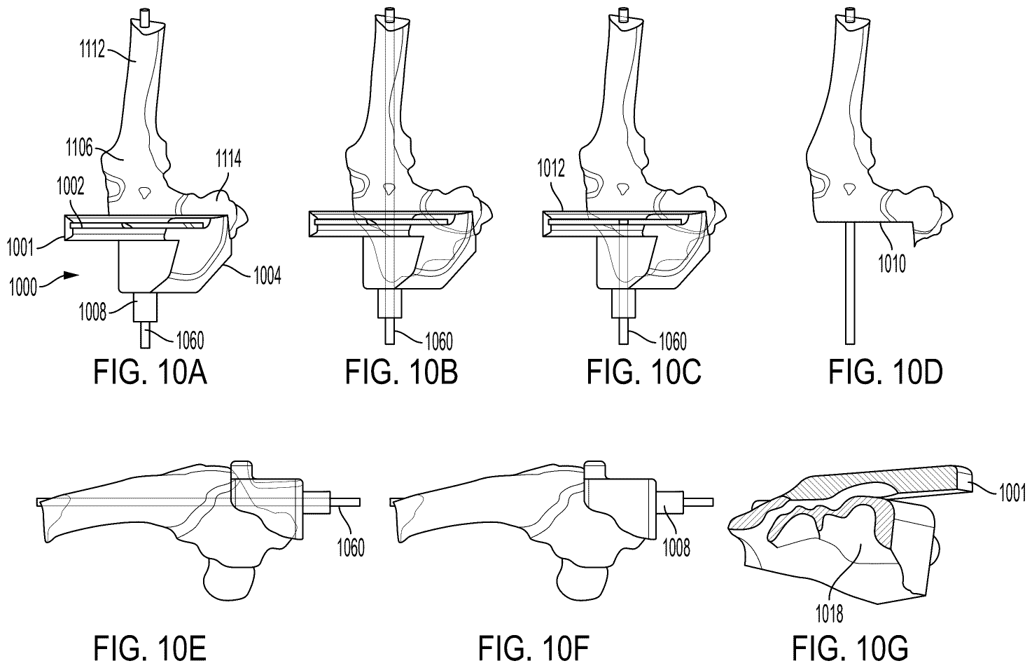

FIGS. 10A-10G illustrate another embodiment of a carpal guide 1000. The carpal guide 1000 includes a carpal guide body having a proximal portion 1006 and a distal portion 1012. The carpal guide 1000 can substantially include one or more (e.g., all) of the features relating to the carpal guides described herein (e.g., carpal guide 300). In addition, as best shown in FIGS. 10A-10C, carpal guide 1000 can include a slot body 1001 having a slot 1002. The Slot 1002 may be used to cut through one or more bones of the patient, such as the capitate bone 1106 and/or the hamate bone 1114 of the patient. Cutting through such bones can create a planar surface, such as a planar surface 1010 (FIG. 10D). One or more implants, such as one or more implant baseplates, can be set upon the cut bone (e.g., set upon the planar position 1010).

In an exemplary embodiment, the slot body 1001 and slot 1002 can be perpendicular to the guidewire 1060. The location (e.g., proximal-distal location) of the slot 1002 can be determined during preoperative planning. In an embodiment, the slot 1002 can include an articular surface 1018 that interfaces with one or more bones of the patient, such as the capitate 1106, hamate 1114, and third metacarpal 1112 of the patient, as well as other bones such as the trapezoid and second metacarpal. The articular surface 1018 can be configured based on the anatomy of the patient and/or the planned position of the bone cut. In an embodiment, the articular surface 1018 of the slot 1002 can be raised off of the trapezoid and second metacarpal of the patient, which may cause the articular surface 1018 to contact (e.g., only contact) the capitate 1106, the hamate 1114, and third metacarpal 1112.

As described herein relating to carpal guide 300, the carpal guide 1000 can be placed on the bone of the patient and pinned with one or more k-wires. The guidewire 1060 can be inserted within an aperture of carpal guide wire housing 1008. The guidewire 1060 can be pulled back in the direction of the proximal portion 1006 prior to the cut being performed upon the bone through the slot 1002.

While the invention has been described with respect to specific examples including exemplary modes of carrying out the subject disclosure, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the subject disclosure should be construed broadly as set forth in the appended claims.

I/we claim:

1. A wrist implant guide assembly comprising:
a radial guide that includes:
a radial guide body having a patient-specific surface for matingly contacting a radial bone of a patient, and a radial guide wire aperture configured to direct a guide wire into a radial bone of the patient; and
a carpal guide that includes:
a carpal guide body having a patient-specific surface for matingly contacting a capitate bone and a hamate bone of the patient, and a carpal guide wire aperture configured to direct a guide wire into a capitate bone of the patient.

2. The wrist implant guide assembly of claim 1, wherein the patient-specific surface of the radial guide matingly corresponds to a distal portion of the radius bone of the patient.

3. The wrist implant guide assembly of claim 1, wherein the radial guide further comprises a dorsal flange extending from the radial guide body having a patient-specific surface for matingly contacting and covering a Lister's tubercle of the patient.

4. The wrist implant guide assembly of claim 3, wherein the dorsal flange includes a plurality of stabilizer apertures.

5. The wrist implant guide assembly of claim 4, wherein the plurality of stabilizer apertures extend in non-parallel and non-perpendicular directions relative to each other.

6. The wrist implant guide assembly of claim 1, wherein the radial guide further comprises a guide wire housing extending from the radial guide body, the guide wire housing including the radial guide wire aperture.

7. The wrist implant guide assembly of claim 1, wherein the radial guide body has a substantially planar distal surface from which a guide wire housing extends.

8. The wrist implant guide assembly of claim 1, wherein the radial guide further comprises a plurality of peripheral radial guide wire apertures positioned peripherally about the radial guide wire aperture.

9. The wrist implant guide assembly of claim 1, wherein the carpal guide further comprises a guide wire housing extending from the carpal guide body, the guide wire housing including the carpal guide wire aperture.

10. The wrist implant guide assembly of claim 1, wherein the carpal guide further comprises a plurality of peripheral carpal guide wire apertures positioned peripherally about the carpal guide wire aperture.

11. The wrist implant guide assembly of claim 1, wherein the carpal guide further comprises an alignment arm for engaging a third metacarpal of the patient.

12. The wrist implant guide assembly of claim 11, wherein the alignment arm extends from the carpal guide body.

13. The wrist implant guide assembly of claim 11, wherein the carpal guide body comprises a stationary base extending therefrom, and the alignment arm comprises a moveable arm engageable with the stationary base.

14. The wrist implant guide assembly of claim 11, wherein the alignment arm has a longitudinal length extending substantially parallel to an axis of the carpal guide wire aperture.

15. The wrist implant guide assembly of claim 1, further comprising a drill guide configured to engage the radial guide or the carpal guide.

16. The wrist implant guide assembly of claim 15, wherein drill guide comprises a barrel that includes a plurality of guide wire apertures extending longitudinally therethrough.

17. The wrist implant guide assembly of claim 15, wherein the drill guide comprises a barrel having an overall longitudinal cross-sectional profile that is non-circular.

18. The wrist implant guide assembly of claim 15, wherein the drill guide comprises a barrel, and wherein the radial guide further comprises a guide wire housing extending from the radial guide body and having a corresponding slot to receive the barrel of the drill guide therein, the barrel configured to engage the slot of the radial guide body.

19. The wrist implant guide assembly of claim 15, wherein the drill guide comprises a barrel, and wherein the carpal guide further comprises a guide wire housing extending from the carpal guide body and having a corresponding slot to receive the barrel therein, the barrel configured to engage the slot of the carpal guide body.

20. A wrist implant guide assembly comprising:
a radial guide that includes:
a radial guide body having a patient-specific surface for matingly contacting a distal portion of the radial bone of a patient, and a substantially planar distal surface,
a radial guide wire aperture configured to direct a guide wire into a radial bone of the patient,
a plurality of peripheral radial guide wire apertures positioned peripherally about the radial guide wire aperture,
a dorsal flange extending from the radial guide body having a patient-specific surface for matingly contacting and covering a Lister's tubercle of the patient, the dorsal flange including a plurality of stabilizer apertures extending in non-parallel and non-perpendicular directions relative to each other, and
a guide wire housing extending from the radial guide body, the guide wire housing including the radial guide wire aperture; and
a carpal guide that includes:
a carpal guide body having a patient-specific surface for matingly contacting a capitate bone and a hamate bone of the patient, and a stationary base extending therefrom,
a guide wire housing extending from the carpal guide body, the guide wire housing including a carpal guide wire aperture configured to direct a guide wire into a capitate bone of the patient,
a plurality of peripheral carpal guide wire apertures positioned peripherally about the carpal guide wire aperture, and
an alignment arm extending from the carpal guide body for engaging a third metacarpal of the patient, the alignment arm including a moveable arm engageable with the stationary base and a longitudinal length extending substantially parallel to an axis of the carpal guide wire aperture.
